(19) United States
(12) Patent Application Publication
(10) Pub. No.: US 2014/0243214 A1
(43) Pub. Date: Aug. 28, 2014

(54) FET ARRAY SUBSTRATE, ANALYSIS SYSTEM AND METHOD

(71) Applicant: Hitachi, Ltd., Chiyoda-ku, Tokyo (JP)

(72) Inventors: Takanobu Haga, Tokyo (JP); Itaru Yanagi, Tokyo (JP); Naoshi Itabashi, Tokyo (JP); Yoshimitsu Yanagawa, Tokyo (JP); Takeshi Ohura, Tokyo (JP); Takashi Anazawa, Tokyo (JP)

(73) Assignee: Hitachi, Ltd., Chiyoda-ku, Tokyo (JP)

(21) Appl. No.: 14/241,041

(22) PCT Filed: Feb. 26, 2013
(86) PCT No.: PCT/JP13/55004

§ 371 Date: Feb. 25, 2014

(51) Int. Cl. G01N 33/487; G01N 27/414

(52) U.S. Cl. CPC G01N 33/48721; G01N 27/4145
USPC 506/2; 257/253; 422/82.02; 436/94; 506/38

(57) ABSTRACT

In an FET configuration having a channel with a small thickness, transistor characteristics vary for different FETs in the same array, and therefore when the same gate voltage is applied, the sensitivities of DNA detection may be insufficient. To this end, the change in the channel current when DNA passes through the nanopore is detected while applying an optimum gate voltage for each nanopore FET to attain a predetermined channel current value to a plurality of nanopore FETs disposed on the same substrate, and four types of bases constituting DNA are distinguished.

### TECHNICAL FIELD

The present invention relates to a semiconductor sensor for nucleic acid analysis and the like, and especially to an analytical technique using an FET array substrate.

### BACKGROUND ART

As a method for determining deoxyribonucleic acid (DNA) sequence without using a reagent, a measurement method using a hole (nanopore) with nanometer sizes which are as large as DNA and a nanopore device with electrodes provided therearound has been drawing attention. Non-patent document 1 and patent document 1 disclose configurations and methods for measuring a change in a current between channels when DNA passes through the nanopore using a field effect transistor (FET) structure produced on a semiconductor substrate and the nanopore. The FET structure has a source electrode, a drain electrode, and a channel (silicon nanowire with a diameter of 20 nm or larger) which connects the two electrodes (refer to FIGS. 1 and 3 of non-patent document 1). In addition, the structure has a nanopore which penetrates the substrate over the channel. The spaces above and below the substrate are filled with an electrolyte, the solution molecules in upper reservoir and lower reservoir can move between the two reservoirs only through the nanopore. When the electrodes are immersed in the two reservoirs and a voltage is applied to the electrodes, an ion current caused by ionic substances which have passed through the nanopore flows.

In addition, when a voltage is applied between the source and drain, a current also flows to the channel (hereinafter referred to as “channel current”). The electrodes immersed in the solution also function as gate electrodes for causing the channel current to flow. When DNA passes through the nanopore, the ion current is blocked and the value decreases (block current), so that the passage of DNA can be known. Simultaneously, the potential around the nanopore varies in correlation with the effective electric charge of nucleotide, and therefore a change in the channel current is also measured. Patent document 1 refers to the possibility of DNA sequence decision by the change in the above channel current. FIG. 5 of patent document 1 illustrates the array configuration of a nanopore FET.

In contrast, FIGS. 5a to 5e of patent document 2 illustrate the configuration in which a gate electrode and further a nanopore are provided in the channel which connects the source and drain. The patent document describes a method for distinguishing four types of bases of DNA which pass through the nanopore by detecting a change in the channel potential around the nanopore caused by a difference in the effective charge of nucleotide when DNA passes through the nanopore as a change in the channel current. The voltage applied to the electrolyte in the upper reservoir divided by the device or the voltage applied to the electrode designated by 70 in FIG. 5c becomes the gate voltage. By applying the gate voltage and forming an inversion layer below the gate, the channel is formed. A current then flows between the source and drain through the channel. The thickness of the inversion layer is very small, and the thickness of a current path has a value almost the same as the size of one base. In FIG. 5d, there is the description that the control gate is provided to the side of the channel and gate, and an appropriate voltage is applied to the control gate, so that the current path gathers closer to the pores in the channel.

### CITATION LIST

### Patent Literature

[Patent document 1] US2010/0327847 A1
[Patent document 2] US2011/0133255 A1

### Non-patent Document

[Non-patent document 1] Pin Xie et al., vol 7, 119-125, Nature Nanotechnology (2011)

### SUMMARY OF THE INVENTION

### Problems to be Solved by the Invention

As stated above, patent document 1 illustrates, in FIG. 5, the array configuration of a nanopore FET, but in the configuration illustrated, electrodes immersed in a solution are shared as gate electrodes, and therefore different gate voltages cannot be applied to the nanopore FETs. Accordingly, the nanopore FETs configuring the nanopore FET array cannot be controlled respectively, and the DNA detection sensitivity of the nanopore FET array cannot be obtained sufficiently.

An object of the present invention is to solve the above-mentioned problems, and to provide an FET array substrate, an analysis system, and a method which are capable of providing sufficient detection sensitivity.

### Solution to Problem

In order to achieve the above-mentioned object, the present invention provides a field effect transistor (FET) array substrate including a source, a drain, a channel and a gate formed on an insulation film, and a through hole or a non-through hole formed on the insulation film and allowing an object to be detected to enter, at least two FETs to which different gate voltages can be applied to exert a field effect on the channel by the gate disposed, the through hole or non-through hole being disposed near the side of the channel, and detecting the presence or absence, or the change of an object to be detected in the through hole or non-through hole from a change in a current flowing from the source to the drain.

Moreover, in order to achieve the above-mentioned object, the present invention provides an analysis system including an FET array substrate having a source, a drain, a channel and a gate formed on an insulation film, a through hole or a non-through hole formed on the insulation film and allowing an object to be detected to enter, at least two FETs to which different gate voltages can be applied to exert a field effect on the channel by the gate disposed, the through hole or non-through hole being disposed near the side of the channel, the FET array substrate detecting the presence or absence, or the change of object to be detected in the through hole or non-through hole from a change in a current flowing from the source to the drain, two solution reservoirs separated by the FET array substrate, two electrodes immersed in the solution reservoirs, a first power source which applies a voltage to the electrodes, a second power source which applies a voltage between the source and drain, and an ampere meter which measures a current flowing through the channel.

Furthermore, in order to achieve the above-mentioned object, the present invention provides an analysis method including immersing an FET array substrate into a solution reservoir, the FET array substrate comprising a source, a drain, a channel, a gate, and a through hole or non-through hole which allows an object to be detected to enter formed on the insulation film, and at least two FETs to which different gate voltages can be applied to exert a field effect on the channel by the gate disposed, the through hole or non-through hole being disposed near the side of the channel, and detecting the presence or absence, or the change of object to be detected in the through hole or non-through hole from a change in a current flowing from the source to the drain, applying a voltage between the source and drain, and measuring a current flowing between the channels.

### Advantageous Effect of the Invention

According to the invention of the present application, the proportion of nanopore FETs which are capable of detecting an object with good sensitivity can be increased. Accordingly, the parallel processing of the measurement of objects to be detected improves.

### BRIEF DESCRIPTION OF THE DRAWINGS

FIG. 1A is a perspective view which shows a constitutional example of the nanopore FET according to a first example.

FIG. 1B is an enlarged perspective view of the vicinity of a nanopore of a constitutional example of the nanopore FET according to the first Example.

FIG. 1C is a drawing for illustrating the characteristics of the nanopore FET according to the first Example.

FIG. 2 is a drawing which shows a configuration of a DNA base sequence measurement system using an FET array substrate according to the first Example.

FIG. 3 is a drawing for illustrating an electric configuration of the FET array substrate according to the first Example.

FIG. 4A is a drawing which shows an example of a flowchart for measuring DNA by applying different gate voltages to the respective nanopore FETs according to the first Example.

FIG. 4B is a drawing which shows an example of a Vc value table for measuring DNA by correcting the characteristics of the nanopore FET according to the first Example.

FIG. 5A is a drawing which shows the graph of the IV characteristics of the respective nanopore FETs according to the first Example before correction.

FIG. 5B is a drawing which shows the graph of the IV characteristics of the respective nanopore FETs after correction according to the first Example.

FIG. 6 is a drawing which shows an example of a configuration of the FET array substrate according to a second Example.

FIG. 7 is a drawing which shows an example of a configuration of the FET array substrate according to a third Example.

FIG. 8A is a perspective view of a constitutional example of a nanopore FET according to a fourth Example.

FIG. 8B is a cross-sectional view of a constitutional example of the nanopore FET according to the fourth Example.

FIG. 9 is a flowchart for measuring DNA by applying a different gate voltage to each non-through nanopore FET of the fourth Example.

### DESCRIPTION OF EXAMPLES

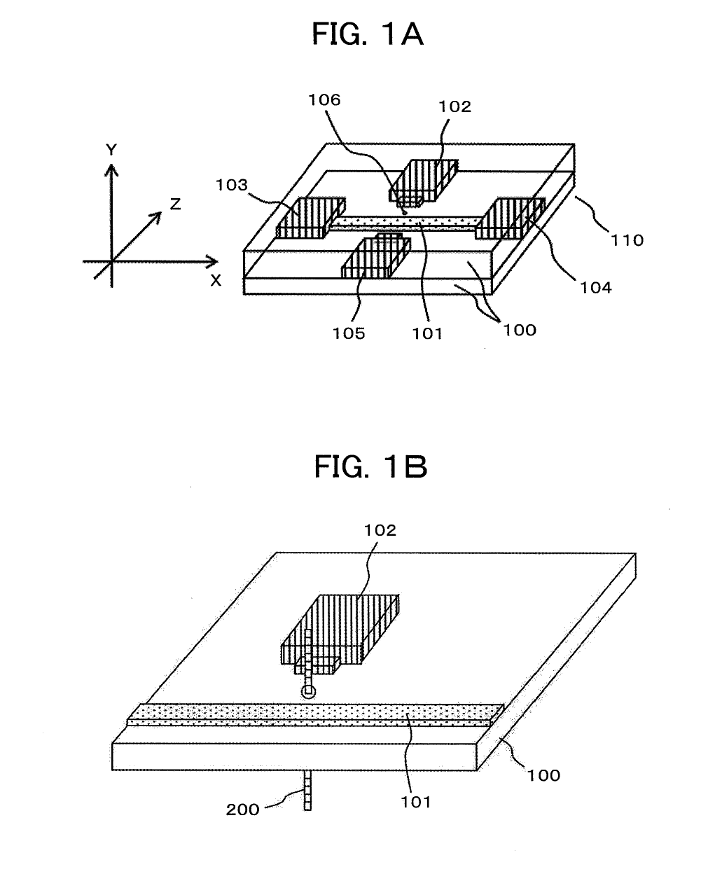

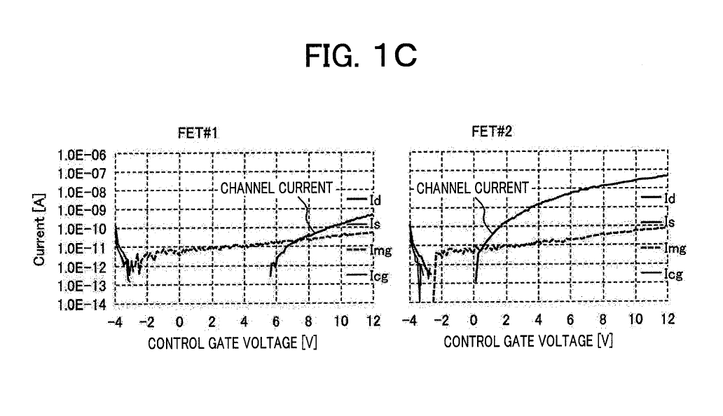

The mode for carrying out the present Invention will be described below. First, the basic configuration of the present invention will be described. That is, a perspective view of the basic configuration of a nanopore FET configuring an FET array substrate for use in detection analysis of an object to be detected for the purpose of determining DNA base sequences and other purposes is shown in FIG. 1A.

In FIG. 1A, 100 is an insulation film, 101 is a channel, 102 is a control gate, 103 is a source, 104 is a drain, 105 is a back gate, and 106 is a nanopore. Additionally, there are four wirings which serve as contacts for the control gate 102, source 103, drain 104, and back gate 105, which are omitted in the figure. The nanopore 106 is between the side face on the channel side of the control gate 102 and the side face on the control gate 102 side of the channel. It should be noted that the insulation film 100 of FIG. 1A is composed of an SiO2 film and an SiN film used for producing normal semiconductors. These insulation films are formed on, for example, a substrate such as Si, or may be an insulation film substrate formed by laminating an SiN film, an SiO2 film and the like.

FIG. 1B shows an enlarged perspective view of the vicinity of the nanopore of the basic configuration. A DNA 200 passes through the nanopore. It should be noted that a series of the blocks of the DNA 200 in the figure represents a series of bases. For example, the channel 101 is a non-doped silicon or a P-type silicon or a low-concentration N-type silicon, while the gates such as the control gate 102 and the back gate 105 are N-type or a P-type silicon (Si), and the source 103 and the drain 104 are N-type silicon (Si).

In this basic configuration, by controlling the control gate voltage in a state that a voltage is applied between the source 103 and the drain 104 so that a source voltage < a drain voltage, this device operates as a transistor. It is a so-called side gate type transistor. When the transistor is in an ON-state, an inversion layer is induced in a channel side portion on the control gate side, that is, the nanopore side, and a current therefore flows in the channel side portion on the control gate side. The thickness of the inversion layer, although varying depending on the control gate voltage, is very thin and is about 2 to 3 nm or less. The thickness of the channel in the Y direction is about 4 nm or less.

The channel current changes depending on the change in the electric field caused by differences in the effective charge and effective field of nucleotide, that is, deoxyribonucleotide triphosphate (dNTP) of four bases of DNA which passes through the nanopore 106, and the identification and decoding of the sequence of the four bases are performed by detecting the change.

However, the inventors of the present invention have found that in an FET configuration having a thin channel with a thickness of about 4 nm or less as mentioned above, the transistor characteristics greatly vary for different FETs in the same array. There are some nanopore FETs in which the sensitivity becomes insufficient when the same gate voltage is applied for the above-mentioned difference.

FIG. 1C is the IV characteristics of an FET having a thickness of the channel in the Y direction of 2 nm. The horizontal axis represents a voltage of the control gate 102, while the vertical axis represents a channel current value. 0 V was applied to the back gate, while 1 V to the drain, and 0 V to the source. As shown in FIG. 10, FET#1 and FET#2 are FETs produced on the same wafer and having the same configuration and same size, but FET#1 showed a rising edge in the channel current value at the control gate voltage of about 6 V, while FET#2 did so at the control gate of about 0 V. In order to detect changes in the channel current when DNA passes through the nanopore with a good signal-noise ratio, it is desirable that a current of nA level flows to the channel. However, in the above-mentioned example, for example, when 4 V is applied evenly to the control gates, a current of nA level flows in FET#2, but the current is 1 nA or lower in FET#1, thereby failing to detect DNA with a good signal-noise ratio.

To this end, the present invention is so configured that the source, drain, channel, and gate are provided in the insulation film of the substrate having the above-mentioned basic configuration; a through hole or a non-through hole is provided from one side to the other side of the same for the insulation film in which the source, drain, channel, and gate are formed two or more field effect transistors which exert an electric field effect on the channel by the gate are disposed and the object to be detected is allowed to enter into the through hole or non-through hole. Moreover, the through hole or non-through hole is preferably so configured that it is disposed between the side face on the channel side in the control gate and the vicinity of the side face on the control gate side in the channel the presence of absence of the object to be detected in the through hole or non-through hole and the change in the object to be detected is detected by the change in the current flowing from the source to drain and different the control gate voltages can be applied to further a plurality of transistors.

This configuration allows increasing the proportion of nanopore FETs which are capable of detecting DNA with a good sensitivity. This improves the parallel processing in DNA sequence measurement.

Various examples of the present invention will be sequentially described below with reference to the drawings. It should be noted that the components having the same functions will be denoted by the same numerals in all drawings for explaining examples, and repeated explanation of the same will be omitted as much as possible. The device structures and materials described in Examples are specific examples for realizing the idea of the present invention, and are not for strictly specifying materials and sizes.

### Example 1

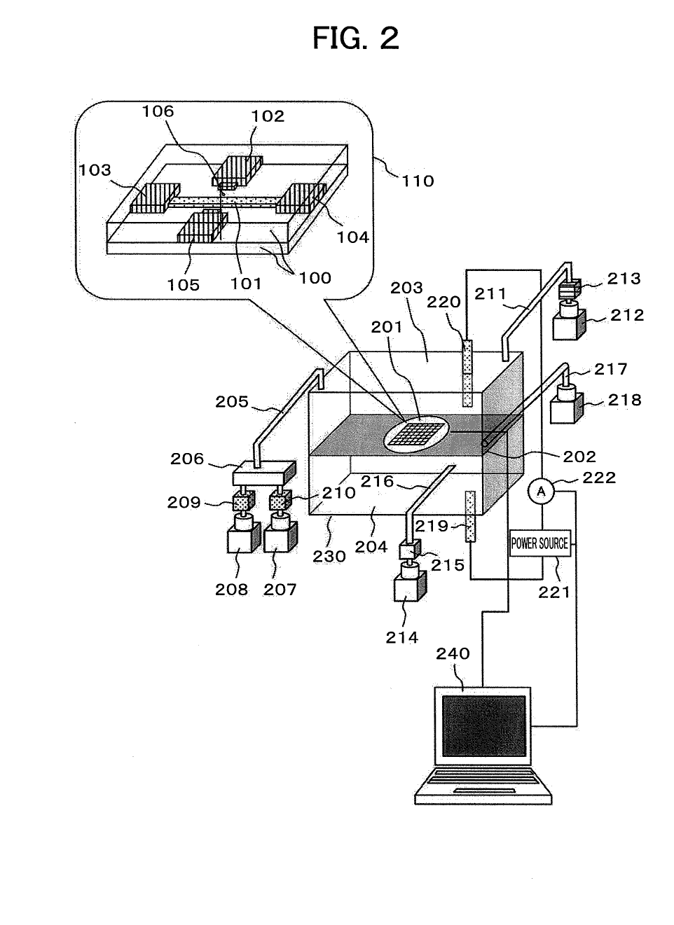

FIG. 2 is a drawing which shows a configuration of a DNA base sequence measurement system using a nanopore FET arrayed substrate (hereinafter referred to as FET array substrate) the according to the first Example.

<Configuration of Flow Cell>

In a flow cell 230, two reservoirs, that is, a solution reservoir c203 and a solution reservoir t204 separated by a partition 202 having an FET array substrate 201 incorporated therein, are provided. On the FET array substrate 201, two or more nanopore FETs 110 are arrayed. Preferably, about 1000×1000=one million of these nanopore FETs are arrayed. The configuration of each of the nanopore FETs 110 is as described with reference to FIG. 1A. An electrode structure, in which the illustration of the control gate 102, the source 103, the drain 104, the back gate 105, the nanopore 106 and other components in the nanopore FET 110 is omitted, is in contact with the solution on the solution reservoir t204 side. The solution in the DNA sample solution container 207 or a buffer container 208 is injected into the solution reservoir c203 by a pump 206 through an injection path 205. Valves 209, 210 are attached to these containers 207 and 208, and the solution to be injected can be selected by opening and closing of the valves 209, 210.

The solution in the solution reservoir c203 is accumulated in a waste liquid container 212 through a discharge path 211. A valve 213 is also attached to the waste liquid container 212 to prevent backward flow. Similarly, a buffer solution is injected by a pump 215 from a buffer container 214 into the solution reservoir t204 through an injection path 216. An excessive waste liquid is discharged into a waste liquid container 218 through a discharge path 217. Although omitted in the figure, the pumps 206, 215 and valves 209, 210, 213 are all connected to a control unit 240, and their operation is automatically controlled. The flow cell 230 is produced by affixing polydimethyl siloxane (PDMS) having flow paths provided thereon on the top and bottom of the partition 202 made of an acrylic resin. The flow paths serve as the solution reservoir c203 and the solution reservoir t204.

<Configuration of Electrodes and Nanopore FETs>

An electrode 220 and an electrode 219 are immersed in the solution reservoir c203 and the solution reservoir t204, respectively. The solution reservoir t204 is filled with the buffer solution. DNA which is the target of decoding of this system floats in the buffer solution in the solution reservoir c203. Since ionic substances are contained in the buffer solution, an ion current generates between the electrode 220 and the electrode 219 by applying a voltage between the two reservoirs. Between the electrode 220 and the electrode 219, a first power source 221 for applying a voltage between the two electrodes and an ampere meter 222 for measuring the ion current value are installed. Moreover, this ampere meter 222 includes an analog-digital (AD) converter.

As shown in FIG. 2, the first power source 221 and the ampere meter 222 are connected to the control unit 240, respectively, and the control unit 240 controls the applied voltage and stores the acquired current value. It goes without saying that the control unit 240 can be composed of a computer provided with a normal central processing unit (CPU), a memory which is a storage unit, an input/output unit such as a keyboard and a display, and a communication interface. The ampere meter 222 can measure the ion current and the blockage current caused when DNA passes through the nanopore DNA. A voltage higher than that of the electrode 220 is applied to the electrode 219. Accordingly, the potential of the solution reservoir t204 becomes higher than that of the solution reservoir c203. Since the DNA floating in the solution reservoir c203 is negatively charged, the DNA passes through the nanopore 106 and moves into the solution reservoir t204. The DNA may be injected not into the solution reservoir c203 but into the solution reservoir t204. In that case, a voltage higher than that of the electrode 219 may be applied to the electrode 220. The ion concentration of the solution reservoir c203 is preferably higher than the ion concentration of the solution reservoir t204. As described in non-patent document 1, the change in the channel current value can be increased (that is, the detection sensitivity can be increased). The ion concentrations of the buffer solution in the solution reservoir c203 and the solution reservoir t204 may be those other than mentioned above.

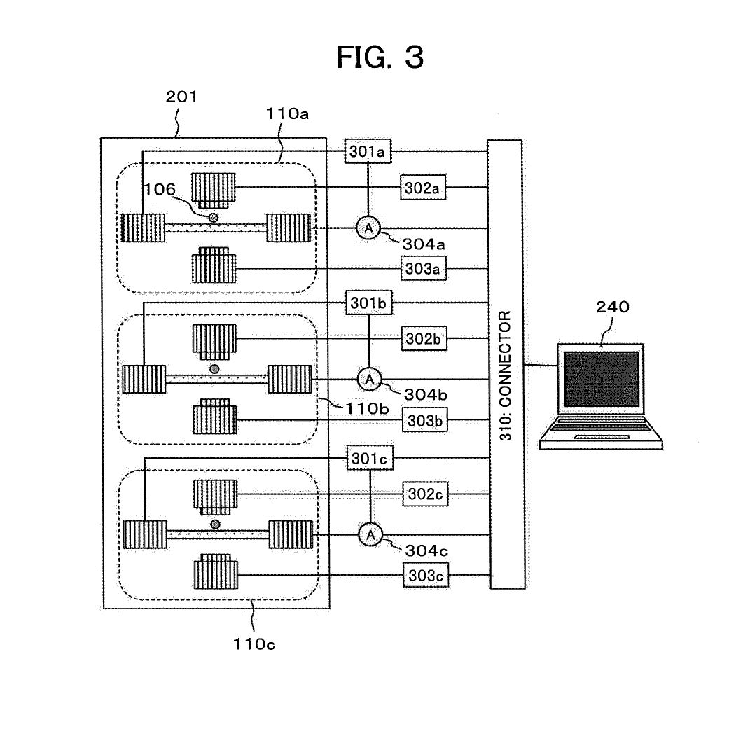

FIG. 3 is a circuit diagram which shows the electric system structure of the FET array substrate 201 of this Example. In this figure, three nanopore FETs 110a, FET 110b, and FET 110c are placed, but the number of the FETs may be any higher than two. Preferably, as previously explained, a number of nanopore FETs are placed. The nanopore FETs are arranged one-dimensionally in the figure, but they may be arranged two-dimensionally. The configuration will be described by taking the nanopore FET 110a as an example. The source is connected to the second power source 301a, the control gate is connected to a power source 302a, and the back gate is connected to a power source 303a. Different voltages are applied independently to these electrodes. The channel current flowing between the source and drain is converted into a digital signal by an amplifier and analog-digital converter (AD converter) incorporated into an ampere meter 304a, and is transmitted to a memory of the control unit 240. All power sources 301a, 302a, 303a, 301b, 302b, 303b, 301c, 302c, 303c and the ampere meters 304a, 304b, 304c are connected to the control unit 240 via a connector 310, and the voltages of the power sources are automatically controlled by the control unit 240.

<Method for Correcting Characteristics of Nanopore FETs>

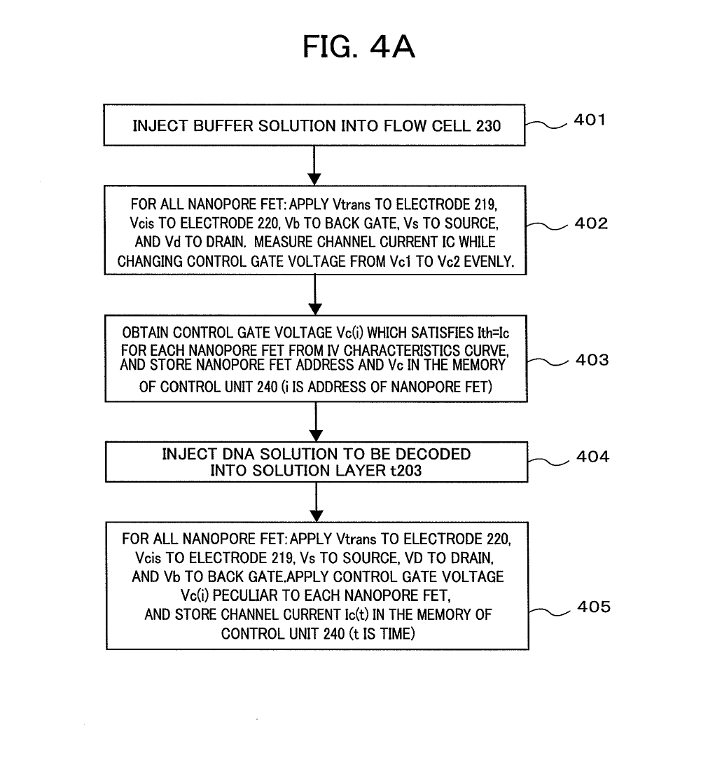

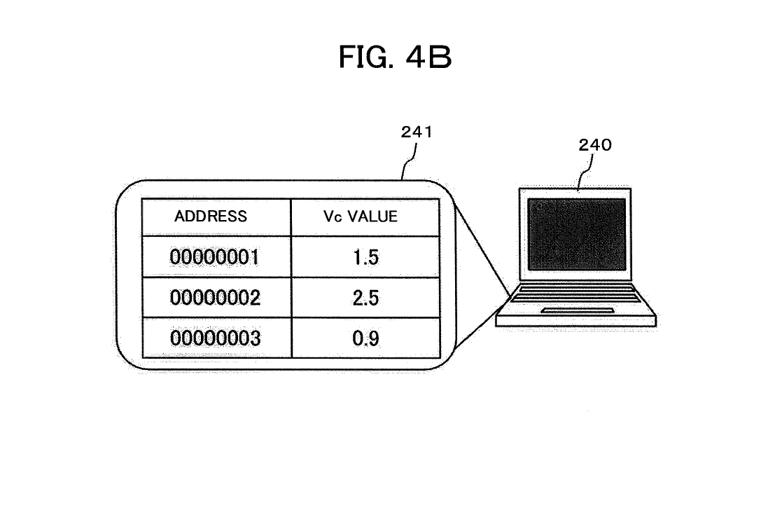

FIGS. 4A and 4B are drawings which show examples, etc., of a flowchart for accurately measuring DNA by applying different gate voltages to the respectively nanopore FETs in the DNA base sequence measurement system in this Example. In order to measure DNA with a good signal-noise ratio, a channel current of a few nA needs to be ensured. The flowchart of FIG. 4A shows an example of a correction method for realizing the same.

In FIG. 4A, a buffer solution which dissolves the DNA to be decoded is first injected into the flow cell (401). Next, the IV characteristics, that is, changes in channel current values Ic when the control gate is changed at a back gate voltage constant value Vb, of all nanopore FETs are measured (402).

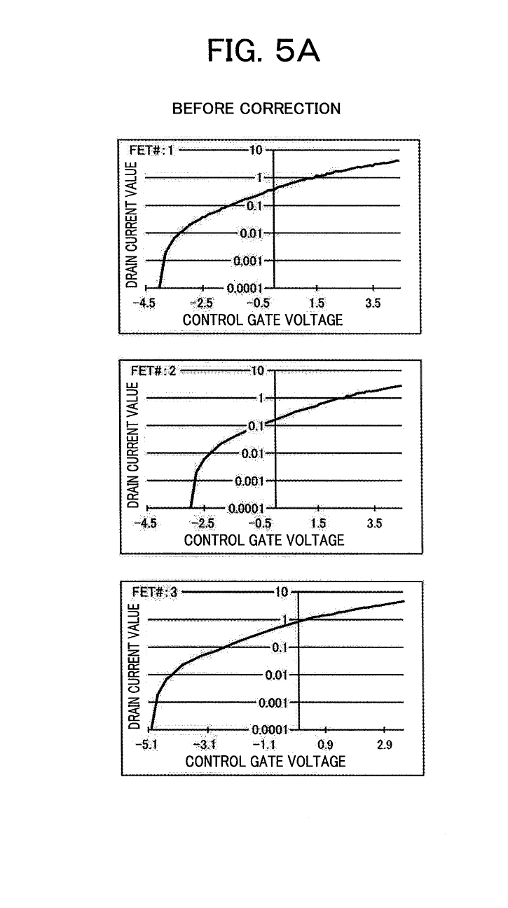

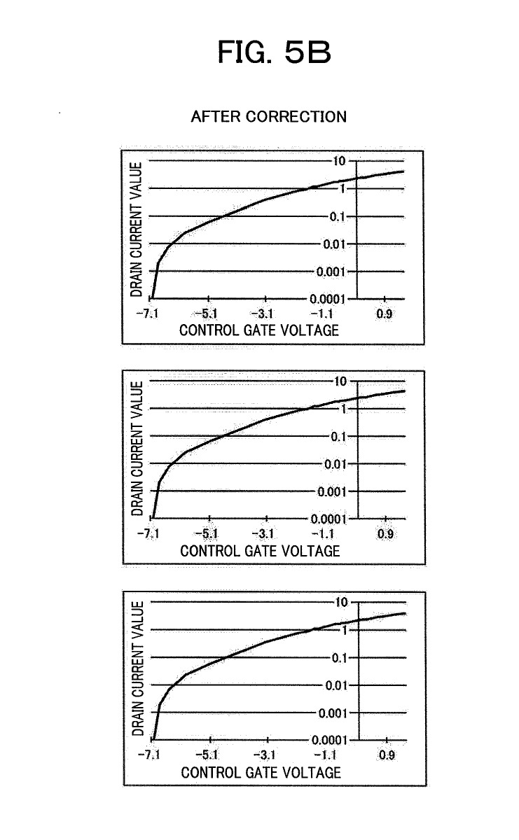

As shown in FIG. 5A, the measured IV characteristics vary for each nanopore FET, and therefore the control gate voltage Vc which attains Ith=1 nA varies for each nanopore FET. This is acquired in step 402 and is stored in a memory 241 in the control unit 240 (403). FIG. 4B shows an example of the value of the control gate voltage Vc stored in a memory which is a storage unit of the control unit 240. As can be seen from this figure, a Vc value is stored in the address corresponding to each of the nanopore FETs.

The solution of the DNA to be decoded is now injected into a solution reservoir t203 (404), and the control gate voltage Vc(i) (i is the address specific to each nanopore FET) is applied, and the channel current is measured (405). Herein, Ith is set to 1 nA, but it is preferably any numerical value from 1 to 10 nA, and may be 11 to 100 nA, and 0.1 to 1 nA. In the example of FIG. 5A, Vtrans=0, Vcis=1 V, Vb=3, Vs=1 V, Vd=0 and the control gate voltage is changed from Vc1=−5.5 to Vc2=4 V, but any voltage can be selected depending on the specification of the nanopore FET. According to the flowchart of FIG. 4A, Vc(00000001)=1.5 V is applied in FET #00000001, Vc(00000002)=2.5 is applied in FET 00000002, and Vc(00000003)=0.9 V is applied in FET#00000003 to measure the channel current when the DNA passes through.

In the above-mentioned example, a specific value is set to the control gate voltage by using the same value for all nanopore FETs to the back gate voltage. Contrary to this example, a value specific to each nanopore FET may be set to the back gate voltage, and the same value may be set to the control gate voltage.

FIG. 5B is the IV characteristics after the correction, that is, when the back gate voltage optimum for each nanopore FET is applied. By applying, as the back gate voltage, 3 V in FET#00000001, 5 V in FET#00000002, and 1.8 V in FET#00000003, correction can be made so that the IV characteristics curves of the three nanopore FETs overlap. In this case, the channel current when the DNA passes through is measured by applying the common control gate Vc=−1.5 V to all nanopore FETs. In addition, in an operation 403 of FIG. 4A, the back gate voltage specific to each nanopore FET is stored along with the address in place of the control gate voltage.

It should be noted that Ith may not be a specific value, but may be a numerical value with a certain range. For example, with Ith ranging from 1 to 10 nA, a current value at which the derivative dV/dI of the IV characteristics curve becomes the greatest in the above range is set to Ic. In this case, there is an effect that the greater the derivative, the more noticeable the difference in the change in the channel current between bases.

In the above example, the correction of the nanopore FET characteristics is performed before the DNA measurement, but it may be performed during the measurement. In this case, the IV characteristics which have changed during the measurement can be also corrected.

### Example 2

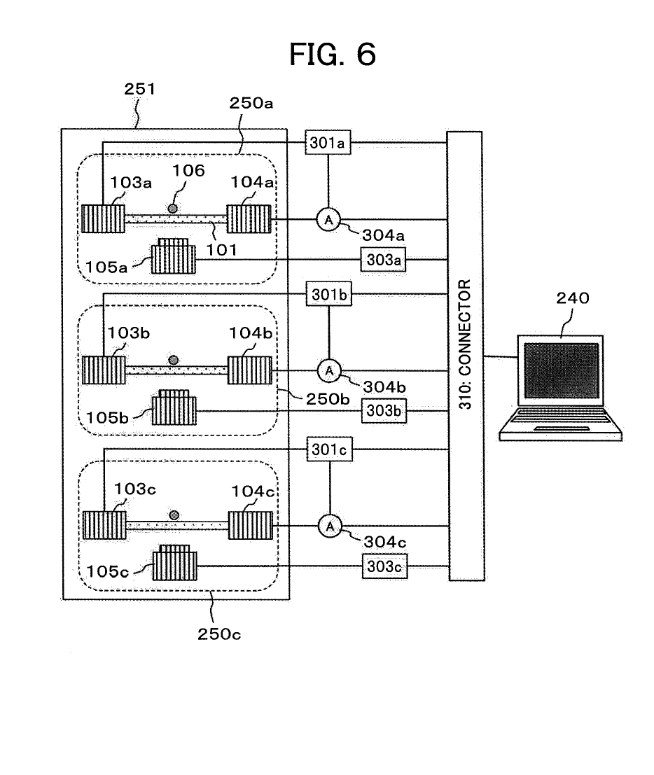

Subsequently, Example 2 of the FET array substrate according to the second Example will be described. FIG. 6 shows an example of the configuration of an FET array substrate 251 in Example 2. The electrodes of nanopore FETs 250a, 250b, 250c in this Example are characterized in that they are composed only of the back gates 105a, 105b, 105c, the sources 103a, 103b, 103c, and the drains 104a, 104b, 104c installed on the side opposite to the side on which the nanopore 106 of the channel is disposed. The configuration of the other components such as the channel 101 and the nanopore 106 is the same as that of the nanopore FETs 110a, 110b, 110c of the FET array substrate 201 of Example 1. Providing no control gate in the configuration of this Example results in a simper configuration than that in Example 1.

It should be noted that the absence of the control gate may lower the channel current value which flows at the same back gate voltage than that in Example 1. In this case, a voltage higher than that in Example 1 may be applied to the electrode 220 to increase the channel current value. In the chart of FIG. 4, the control gate may be carried out as a back gate. The address of each nanopore FET and the specific back gate voltage Vb are stored in the memory.

### Example 3

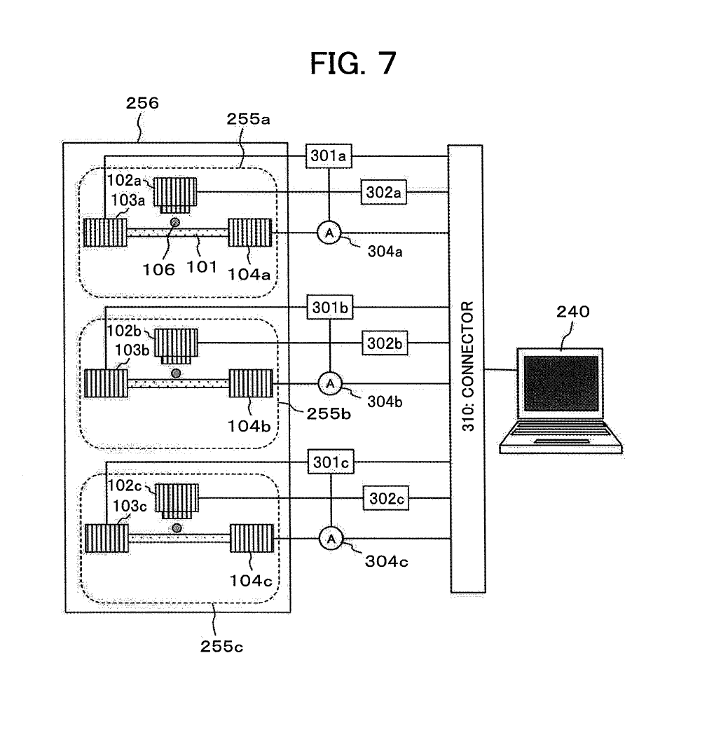

Next, Example of the FET array substrate according to the third Example will be described. FIG. 7 is a drawing which shows an example of the configuration of the FET array substrate according to Example 3. In the configuration of an FET array substrate 256 in Example 3, the electrodes of nanopore FETs 255a, 255b, 255c are characterized in that they are composed of the control gates 102a, 102b, 102c, the sources 103a, 103b, 103c, the drains 104a, 104b, 104c. The configuration other than this is the same as in Example 1. In this Example, as in Example 2, a voltage higher than that in Example 1 may be applied to the electrode 220 to increase the channel current value. The DNA measurement is performed according to the chart of FIG. 4A. Providing no back gate in this Example achieves a simpler configuration than that in Example 1. In addition, the current channel in the channel can be approached to the nanopore, leading to a high sensitivity.

### Example 4

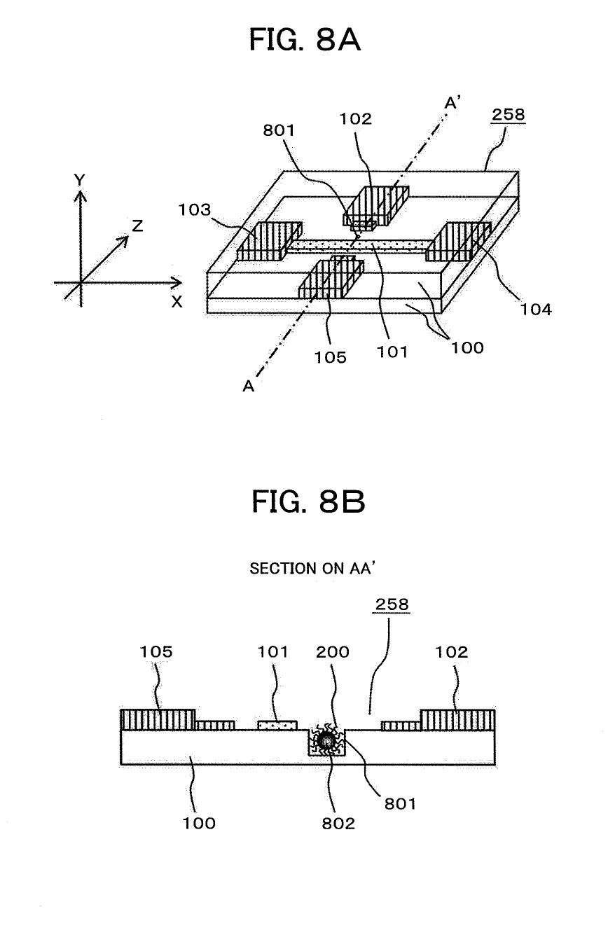

Furthermore, Example of the FET array substrate according to Example 4 will be described. FIGS. 8A and 8B are drawings which show a constitutional example of a nanopore FET 258 in Example 4. In Example 1 and other examples described previously, explanation has been provided on the premise that the nanopores on the nanopore FET are through-nanopores. As for the nanopore FET 258 of this Example, a non-through nanopore is used. FIG. 8A is a block diagram the nanopore FET 258 seen from a diagonal direction of this Example, while FIG. 8B is a cross-sectional view on AA′ of the nanopore FET 258 of FIG. 8A. In the configuration of this Example, as shown in FIG. 8B, the nanopore is characterized in that it is a non-through nanopore 801.

The DNA base sequence measurement system of this Example is almost identical to FIG. 2, but the solution reservoir c204, electrode 220, and the electrode 219 are unnecessary in this Example, while the solution reservoir t203 is only necessary. Furthermore, four containers containing deoxyribonucleotide triphosphates (dATP, dTTP, dCTP, dGTP), respectively, are added, and as the buffer container 208 and DNA sample solution container 207, the above four types of deoxyribonucleotide triphosphates (dNTP) are successively transferred into the solution reservoir t203 by the opening and closing of the valves and the pump drive as described in FIG. 2.

A particle 802 on which a plurarity of the DNA 200 are fixed to be decoded is fixed on the bottom face of the non-through nanopore 801. The DNAs all have the identical sequence. Such a particle 802 is produced in an amplification step by emulsion PCR (Polymerase Chain Reaction). A different amplification step may be used. In order to determine the DNA sequence, base elongation is performed on the particle, and the presence or absence of elongation is detected from a change in the channel current caused by ions released at that time. That is, the DNA sequence can be determined also in the DNA base sequence measurement system of this Example. It should be noted that the above elongation method and similar sequence decision method are described in Jonathan M Rothberg et al. (Nature 2011, doi:10.1038/nature10242).

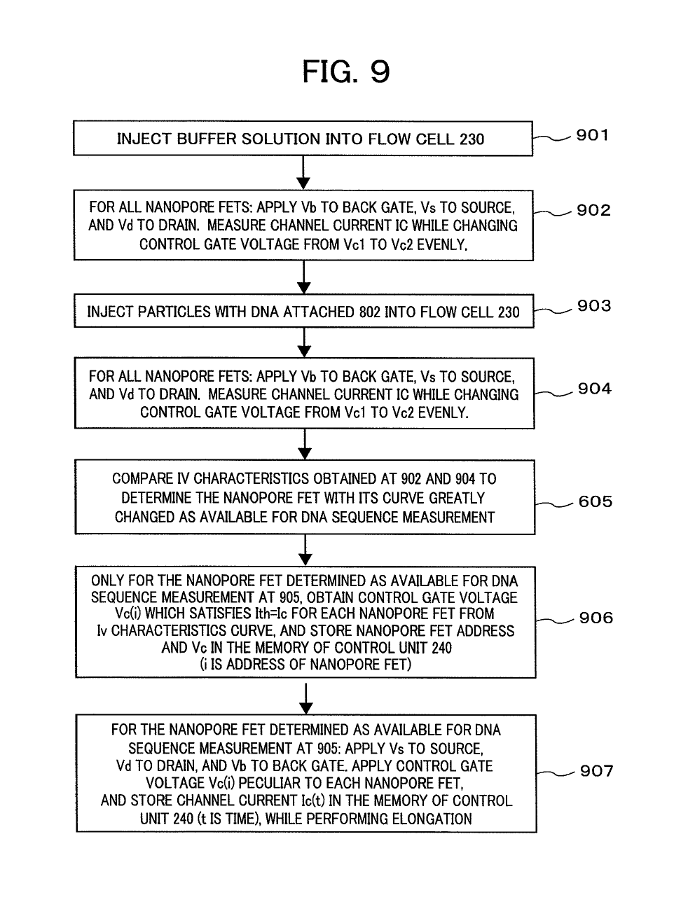

FIG. 9 shows an example of a flowchart for measuring DNA by applying a different gate voltage to each non-through nanopore FET of this Example. This operation is performed before the DNA measurement.

In the flowchart of FIG. 9, first, the buffer solution is injected into the flow cell 230 (901). Moreover, for all nanopore FETs, Vb is applied to the back gate, Vs to the source, and Vd to the drain. The channel current Ic is measured while changing the control gate voltage from Vc1 to Vc2 evenly (902).

Subsequently, the DNA fixed particle 802 is injected into the flow cell 230 (903), and for all nanopore FETs, Vtrans is applied to the electrode 220, Vcis to the electrode 219, Vb to the back gate, Vs to the source, and Vd to the drain. The channel current Ic is measured while changing the control gate voltage from Vc1 to Vc2 evenly (904).

Moreover, a nanopore FET with a curve which has been greatly changed is determined as available for DNA sequence measurement by comparing the IV characteristics acquired in steps 902 and 904 (905). Only with a nanopore FET determined as available for DNA sequence measurement in this step 905, the control gate voltage Vc(i) which attains Ith=Ic is acquired from the Iv characteristics curve for each nanopore FET, and the nanopore FET address and Vc are stored in the memory of the control unit 240 (herein, i represents the address of the nanopore FET) (906).

Furthermore, with the nanopore FET determined as available for DNA sequence measurement in step 905, Vs is applied to the source, Vd to the drain, and Vb to the back gate, the control gate voltage Vc(i) specific to each nanopore FET is applied, and elongation is performed while the channel current Ic(t) is stored in the memory of the control unit 240 (t is time) (907).

In this Example, by performing the operation described above, not only the correction of the FET characteristics is performed, but also the non-through nanopore 801 containing no beads or no DNA fixed thereonto can be known in advance. It is not necessary to acquire the sequence information of DNA from such a non-through nanopore, and therefore an extra amount of data can be decreased.

Moreover, in the above example, released ions were detected from a plurality of the same DNA fragments using the particle 802. Signals are obtained from a number of molecules, which leads to an increased signal-noise ratio. A single DNA may be fixed on the bottom face of the non-through nanopore 801 without using the particle, and the DNA sequence may be determined by elongation. The diameter of the above non-through nanopore 801 is adjusted depending on the size of the object to be fixed to. It should be noted that the process flow of this Example can be carried out in the FET array substrate 201 in any of Examples 1 to 3 with the through nanopore replaced with a non-through nanopore. Since the solution reservoir c204 is unnecessary, the configuration of the device can be made simpler.

It should be noted that the present invention is not limited to Examples mentioned above, and include various variants. For example, the above-described Examples are detailed explanation provided for better understanding of the present invention, and are not for limiting the present invention to those provided with all the configurations explained. Moreover, part of the configuration of certain Example can be replaced with the configuration of another Example, while the configuration of another Example can be added to part of the configuration of certain Example. Moreover, another configuration may be added to, removed from, or replaced with part of the configuration of each of Examples.

Furthermore, the case where the configurations, functions, processes and the like described above are realized by means of software by creating a program which realizes part or all of them has been mainly explained, but it goes without saying that they can be realized by means of hardware, for example, an integrated circuit designed accordingly.

### REFERENCE SIGNS LIST

100 Insulation film
101 Channel
102 Control gate
103 Source
104 Drain
105 Back gate
106 Nanopore
200 DNA
110, 110a, 110b, 110c, 250a, 250b, 250c, 256a, 256b, 256c, 258 Nanopore FET
201, 251, 256 FET array substrate
202 Partition
203 Solution reservoir c
204 Solution reservoir t
205, 216 Injection path
206, 215 Pump
207 DNA sample solution container
208 Buffer container
209, 210, 213 Valve
211, 217 Discharge path
212, 218 Waste liquid container
219, 220 Electrode
221, 302a, 302b, 302c, 303a, 303b, 303c Power source
222, 304a, 304b, 304c Ampere meter
230 Flow cell
240 Control unit
241 Memory
310 Connector
801 Non-through nanopore
802 Particle

1. A field effect transistor (FET) array substrate comprising:
a source, a drain, a channel and a gate formed on an insulation film, and a through hole or a non-through hole formed on the insulation film and allowing an object to be detected to enter, and at least two FETs to which different gate voltages can be applied to exert an electric field effect on the channel by the gate disposed,
the through hole or non-through hole being disposed in the vicinity of the side face of the channel, the FET array substrate detecting the presence or absence, or a change of the object to be detected in the through hole or non-through hole from a change in a current flowing from the source to the drain.

2. The FET array substrate according to claim 1,
wherein the gate comprises a control gate to which a control gate voltage is applied,
the through hole or non-through hole is disposed between a side face of the control gate on the channel side and a side face of the channel on the control gate side.

3. The FET array substrate according to claim 1,
wherein the gate comprises a back gate to which a gate voltage is applied,
the back gate is installed to the side opposite to that on which the through hole or non-through hole of the channel is disposed.

4. The FET array substrate according to claim 2, wherein the gate further comprises a back gate installed on the site opposite to that of the control gate across the channel.

5. The FET array substrate according to claim 2, wherein different voltages are applied to the control gates so that the background current values flowing to the channel become almost the same between a plurality of the FETs.

6. The FET array substrate according to claim 5, wherein the object to be detected is deoxyribonucleic acid (DNA).

7. An analysis system comprising:
a source, drain, channel and gate formed on an insulation film, a through hole or a non-through hole formed on the insulation film and allowing an object to be detected to enter, and at least two FETs to which different gate voltages can be applied to exert an electric field effect on the channel by the gate disposed,
the through hole or non-through hole being disposed in the vicinity of the side face of the channel, an FET array substrate detecting the presence or absence, or a change of the object to be detected in the through hole or non-through hole from a change in a current flowing from the source to the drain,
two solution reservoirs separated by the FET array substrate, and
two electrodes immersed in the solution reservoirs,
a first power source which applies a voltage to the electrodes,
a second power source which applies a voltage between the source and the drain, and
an ampere meter which measures a current flowing through the channel.

8. The analysis system according to claim 7, wherein different voltages are applied to the control gate so that background current values flowing to the channels become almost the same between a plurality of the FETs.

9. The analysis system according to claim 8, wherein the gate comprises a control gate to which a control gate voltage is applied, and the through hole or non-through hole is disposed between a side face of the control gate on the channel side and a side face of the channel on the control gate side.

10. The analysis system according to claim 9, wherein the object to be detected is deoxyribonucleic acid (DNA).

11. The analysis system according to claim 10,
wherein the system further comprises a control unit having a storage unit,
the control unit causes the control gate voltage to change in a predetermined range to measure a channel current, and
determines the control gate voltage to attain a predetermined channel current value for each transistor and store in the storage unit.

12. An analysis method comprising
a source, a drain, a channel, a gate, and a through hole or non-through hole which allows an object to be detected to enter formed on the insulation film, and at least two FETs to which different gate voltages can be applied to exert an electric field effect on the channel by the gate disposed, the through hole or non-through hole being disposed in the vicinity of the side face of the channel, and the FET array substrate detecting the presence or absence, or a change of the object to be detected in the through hole or non-through hole from a change in a current flowing from the source to the drain being immersed in a solution reservoir, and measuring a current flowing between the channels a voltage by applying between the source and the drain.

13. The analysis method according to claim 12, wherein different voltages are applied to the control gate so that background current values flowing to the channels become almost the same between a plurality of the FETs.

14. The analysis method according to claim 13,
wherein the control gate voltage is caused to change in a predetermined range to measure a channel current, and
the control gate voltage to attain a predetermined channel current value is determined for each transistor and applied.

15. The analysis method according to claim 14, wherein the object to be detected is deoxyribonucleic acid (DNA).
